(19) United States
(12) Patent Application Publication
(10) Pub. No.: US 2015/0087725 A1
(43) Pub. Date: Mar. 26, 2015

(54) Injectable Gelling Material

(71) Applicant: Hong Kong Universit of Science and Technology, Hong Kong (HK)

(72) Inventors: Ying Chau, Hong Kong (CN); Yu Yu, Hong Kong (CN)

(21) Appl. No.: 14/361,780

(22) PCT Filed: Nov. 29, 2012
(86) PCT No.: PCT/CN2012/001596

§ 371 Date: May 30, 2014

Related U.S. Application Data
(60) Provisional application No. 61/629,991, filed on Dec. 2, 2011.

(51) Int. Cl. A61L 27/18

(52) U.S. Cl. CPC A61L 27/18; A61L 2430/34; A61L 2400/06
USPC 514/777

(57) ABSTRACT

A material is described herein. The material includes a multi-vinylsulfone containing molecule and a multi-nucleophile containing molecule. The material can form a hydrogel from an aqueous solution with dissolved multi-vinylsulfone and a multi-nucleophile. The aqueous solution can undergo gelation with a controllable gelation time to form the hydrogel upon administration to a space in a body. The hydrogel can form in situ with controllable properties, which allow the hydrogel to be used in many biomedical applications. Examples of biomedical applications include, but are not limited to, a volume/filler expander, a storage and delivery mechanism for biologics, and as a tissue interacting surface.

### CROSS REFERENCE TO RELATED APPLICATIONS

This application claims priority to U.S. provisional patent application no. 61/629,991, entitled: “Uses of Biocompatible in situ Gelling Agents,” and filed on Dec. 2, 2011.

### TECHNICAL FIELD

This disclosure generally relates generally to a material that can form a hydrogel upon injection to a space in the body and to applications of the material.

### BACKGROUND

The term “hydrogel” is used to define a system of water soluble polymers, water insoluble cross linking points, and an aqueous solution that bathes the polymers. The components of the system can be viewed macroscopically as a unit. The water soluble polymers are cross linked by a chemical bond at the cross linking points so that the water soluble polymers are no longer soluble in the aqueous solution. Even though the cross linked polymers are no longer soluble in the aqueous solution, but not precipitated from the aqueous solution, either, which allows the hydrogel to be able to hold a large volume of the aqueous solution, while still maintaining its shape.

The above-described background is merely intended to provide an overview of contextual information regarding hydrogels, and is not intended to be exhaustive. Additional context may become apparent upon review of one or more of the various non-limiting embodiments of the following detailed description.

### SUMMARY

The following presents a simplified summary of the specification in order to provide a basic understanding of some aspects of the specification. This summary is not an extensive overview of the specification. It is intended to neither identify key or critical elements of the specification nor delineate any scope of particular embodiments of the specification, or any scope of the claims. Its sole purpose is to present some concepts of the specification in a simplified form as a prelude to the more detailed description that is presented later.

In accordance with one or more embodiments and corresponding disclosure, various non-limiting aspects are described in connection with an injectable gelling material (e.g., an injectable hydrogel). Two polymers dissolved in a biocompatible aqueous environment can form a hydrogel with controllable properties upon injection to a site in a body. The hydrogel can form in situ with the controllable properties, which allow the hydrogel to be used in many biomedical applications. Examples of biomedical applications include, but are not limited to, a volume/filler expander, a storage and delivery mechanism for biologics, and as a tissue interacting surface.

A material is described, according to an embodiment. The material includes a multi-vinylsulfone containing molecule and a multi-nucleophile containing molecule. The material dissolves in a buffer solution to form an aqueous solution, which can undergo gelation upon administration to a space in a body with a controllable gelation time, swelling ratio, degradation time and a mechanical property to facilitate filling the space.

A method is described, according to a further embodiment. A multi-vinylsulfone containing molecule and a multi-nucleophile containing molecule are dissolved in water to form an aqueous solution. The aqueous solution is injected to a space in a body. A hydrogel is formed at the space after a gelation time elapses to facilitate filling the space.

In another embodiment, a method is described. An aqueous solution is formed by dissolving a multi-vinylsulfone containing molecule and a multi-nucleophile containing molecule in water. The aqueous solution is administered to a site in a body. A biocompatible surface is created upon administration to the site. The biocompatible surface can interact with the body at the space.

The following description and the drawings set forth certain illustrative aspects of the specification. These aspects are indicative, however, of but a few of the various ways in which the various embodiments of the specification may be employed. Other aspects of the specification will become apparent from the following detailed description of the specification when considered in conjunction with the drawings.

### BRIEF DESCRIPTION OF THE DRAWINGS

Numerous aspects and embodiments are set forth in the following detailed description, taken in conjunction with the accompanying drawings, in which like reference characters refer to like parts throughout, and in which:

FIG. 1 is an example non-limiting diagram illustrating injection of an aqueous solution to a site in the body to facilitate formation of a hydrogel, according to an embodiment;

FIG. 2 is an example non-limiting process flow diagram of a method for facilitating the formation of a hydrogel at a site in the body, according to an embodiment;

FIG. 3 is an example non-limiting process flow diagram of a method for forming the aqueous solution that can facilitate the formation of a hydrogel at a site in the body, according to an embodiment;

FIG. 4 is an example non-limiting process flow diagram of method for utilizing an injectable hydrogel as a volume filler/expander, according to an embodiment;

FIG. 5 is an example non-limiting process flow diagram of a method for utilizing an injectable hydrogel to facilitate biologic storage and/or delivery, according to an embodiment;

FIG. 6 is an example non-limiting process flow diagram of a method for utilizing an injectable hydrogel as a tissue interacting surface, according to an embodiment;

FIG. 7 is an exemplary non-limiting representative image of NIH 3T3 cells after incubating with hydrogel, according to an embodiment;

FIG. 8 is an exemplary non-limiting representative live/dead assay image of cells cultured within a hydrogel, according to an embodiment;

FIG. 9 is an exemplary non-limiting illustration showing that mouse skin cells tolerate the hydrogel, according to an embodiment;

FIG. 10 is an exemplary non-limiting table illustrating the possibility of achieving different gelation times using different combinations of parameters, according to an embodiment;

FIG. 11 is an exemplary non-limiting illustration of the swelling of a hydrogel made from mixing HA-VS and HA-SH of DM 20%, according to an embodiment;

FIG. 12 is an exemplary non-limiting illustration of the swelling of a hydrogel made from mixing HA-VS of DM 8% and dex-SH of DM 4%, according to an embodiment;

FIG. 13 is an exemplary non-limiting graph illustrating in vivo swelling and longevity of two formulations compared with commercially available fillers, according to an embodiment;

FIG. 14 shows an exemplary non-limiting illustration of the control of a mesh size of a hydrogel made from mixing HA-VS of DM 8% and dex-SH of DM 4%, according to an embodiment;

FIG. 15 shows exemplary non-limiting illustration of the control of a mesh size of a hydrogel made from mixing HA-VS and HA-SH of DM 20%, according to an embodiment;

FIG. 16 is an exemplary non-limiting NIR image of NIR-IgG encapsulated hydrogel injected subcutaneously into a mouse, according to an embodiment;

FIG. 17 is an exemplary non-limiting illustration comparing the degradation of HA based in situ hydrogels compared to commercially available dermal filter hydrogels, according to an embodiment;

FIG. 18 is an exemplary non-limiting graph showing the storage modulus of hydrogels of different compositions, according to an embodiment; and

FIG. 19 is an exemplary non-limiting representative live/dead assay image of a cell cluster on a HA based hydrogel, according to an embodiment.

### DETAILED DESCRIPTION

Various aspects or features of this disclosure are described with reference to the drawings, wherein like reference numerals are used to refer to like elements throughout. In this specification, numerous specific details are set forth in order to provide a thorough understanding of this disclosure. It should be understood, however, that the certain aspects of disclosure may be practiced without these specific details, or with other methods, components, molecules, etc. In other instances, well-known structures and devices are shown in block diagram form to facilitate description and illustration of the various embodiments.

Hydrogels are useful in many biomedical applications. When a hydrogel is placed into the body, it creates three “spaces:” an “expanded space,” a “storage space,” and an “interfacial space.” Each of the three “spaces” can be utilized in different applications.

The “expanded space” is caused by the bulk hydrogel. Accordingly, the hydrogel can be utilized as a volume filler/expander. The bulk hydrogel creates a space between tissues due to the insertion of the extra volume of the bulk hydrogel. Examples of applications that utilize this property of a hydrogel include: tissue augmentation, viscosupplementation and viscosurgery. One major problem with using a preformed hydrogel for these applications is placing the hydrogel into the body. Because the hydrogel is a volume filler/expander, an invasive surgical procedure is needed to place the hydrogel in the body. The surgical procedure may not be suitable for applications that are solely for aesthetic purposes or for applications that require placing the extra volume at a deep space inside the body.

This problem may be circumvented by grinding the hydrogel into small (e.g., millimeter sized) particles. However, gel particles behave differently than bulk hydrogel. For example, the mechanical properties, the degradation rate, and other properties are different for gel particles compared to bulk hydrogel. Another potential circumvention technique is by using a large gauge needle, but the large gauge needle is invasive and can be painful. Furthermore, for some applications (e.g., filling an aneurism bulge) it is not possible to use large needles.

The “storage space” is present within the hydrogel and is created by the cross linked network and the aqueous medium. Because of the aqueous environment and the hydrophilic nature of the polymer, protein, cells as well as many other labile microscopic biologics can be stored in this space. Accordingly, the hydrogel can be utilized for biologics storage and delivery. Examples of applications that utilize this property of a hydrogel include: drug delivery and tissue engineering. These applications often require the encapsulation of biologics to be mild; however, many crosslinking reactions are harsh and not suitable for labile biologics. Moreover, it may be difficult to store the pre-encapsulated gel because the properties of the biologics and gels may change during the storage time. For example, the drug stored in the gel may diffuse out and/or the cells stored may grow and degrade the gel scaffold.

The “interfacial space” is created between the hydrogel and the body. Because the hydrogel is in close contact with the body, the surface of the hydrogel can be engineered to create suitable interactions between the hydrogel and the adjacent tissues. Accordingly, the hydrogel can be utilized as a tissue interacting surface. Examples of applications that utilize this property of a hydrogel include: wound healing, anti-adhesion applications and tissue adhesion applications.

In accordance with one or more embodiments of this disclosure, described herein is an injectable hydrogel that can form in situ with controllable properties. The injectable hydrogel that forms upon injection of an aqueous solution to a site/space in the body is superior to particles of already-formed particles and need not be injected with a large gauge needle. The injectable hydrogel can be used in biomedical applications at least because it is biocompatible, easily injectable, and has a controllable gelation time. Additionally, the hydrogel also exhibits at least an adjustable swelling, an adjustable mesh size, and an easy surface functionalization. These properties allow the hydrogel to make an excellent material for use as a volume/filler expander, as a storage and delivery mechanism for biologics, and as a tissue interacting surface.

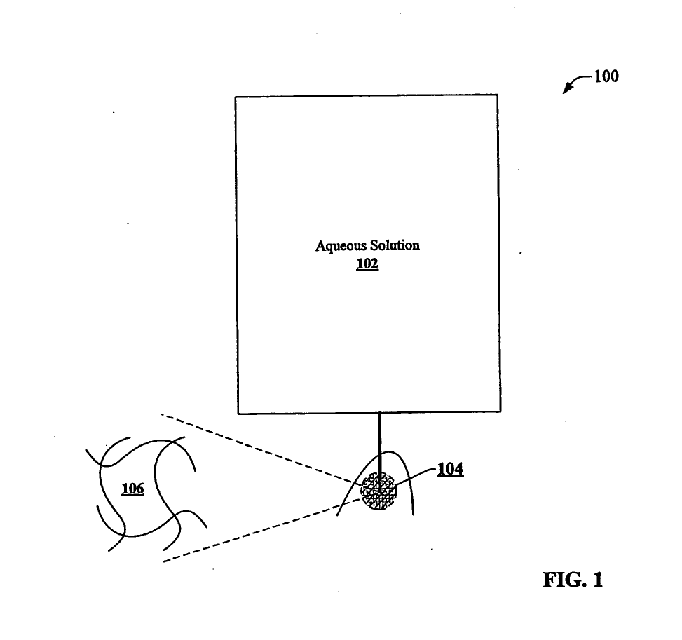

Referring now to the drawings, with reference initially to FIG. 1, illustrated is an example non-limiting illustration 100 of an injection of an aqueous solution to a site in the body to facilitate formation of a hydrogel (also referred to as an “injectable hydrogel”), according to an embodiment. An aqueous solution 102 containing hydrogel precursors can be injected to a spot/site 104 in the body. Upon injection into the site, the aqueous solution 102 can undergo gelation to form a hydrogel 106. Accordingly, the hydrogel 106 is an in situ chemical crosslinkable formulation. The term in situ chemical crosslinkable formulation generally refers to a hydrogel that can be crosslinked at a condition close to physiological conditions.

In an embodiment, the physiological conditions include:

| | |
|---|---|
| Temperature | about 10 degrees Celsius to about 60 degrees Celsius; |
| pH | about 5 to about 9; and/or |
| Water Content | at least about 50 percent. |

In another embodiment, the physiological conditions include:

| | |
|---|---|
| Temperature | about 30 degrees Celsius to about 40 degrees Celsius; |
| pH | about 6.5 to about 8; |
| Water Content | at least about 80 percent. |

In a further embodiment, the physiological conditions include:

| | |
|---|---|
| Temperature | about 35 degrees Celsius to about 39 degrees Celsius; |
| pH | about 7.0 to about 8.0; |
| Water Content | at least about 90 percent. |

In other words, the hydrogel 106 can be formed at a space/site 104 in the body from precursor molecules that are injected into the space/site 104 as an aqueous solution. The precursor molecules, according to an embodiment, are two or more different molecules that can facilitate formation of a hydrogel. The hydrogel must not cause undesirable reaction in the body. Accordingly, the two or more precursor molecules must be biocompatible and non-immunogenic. Examples of biocompatible polymers whose derivatives can be utilized as precursor molecules include hyaluronic acid (HA), polyethylene glycol (PEG), dextran, carboxymethyl cellulose (CMC), polyvinyl alcohol (PVA), alginate, cyclodextran and the like.

In an embodiment, the precursor molecules include one or more biocompatible polymer molecules modified with multiple vinylsulfone groups (multi-vinylsulfone molecule, P-VS) and one or more polymer molecules modified with multiple nucleophile (multi-nucleophile molecule, P-Nu). The P-VS can be, for example, a hydroxyl-bearing biocompatible polymer modified with at least two functional vinylsulfone groups. The vinylsulfone groups can be linked to the hydroxyl group. Examples of nucleophiles include thiols and amines, which can be added to a biocompatible polymer.

When the P-VS and the P-Nu are mixed in an aqueous solution (e.g., a buffer) at a physiological pH, a hydrogel is formed. The vinylsulfone groups are chemically reactive with the nucleophile groups. For example, the vinylsulfone groups can covalently bond to the nucleophile groups to facilitate formation of the hydrogel.

The precursor molecules can dissolve in a biocompatible buffer solution (e.g., water, PBS, or the like) to form the aqueous solution 102. For example, the buffer solution can have a physiological pH. The aqueous solution 102 can also include a salt, a solvent, any other molecule that regulates the pH of the solution, any other molecule that facilitates modification of the precursor polymer, and therapeutic molecule, or any other molecule that minimizes the potential hazard when the precursors or the hydrogel are used in a biological system. The aqueous solution 102 is a viscous solution before gelation so that it can be easily injected to the site/space 104.

Properties of the hydrogel, such as mechanical properties, degree of swelling, mesh size, gelation time, degradation time, and the like, can be tailored or engineered for different purposes. For example, the properties are controllable based on the pH of the aqueous solution, a temperature of the aqueous solution, a concentration of the P-VS within the aqueous solution 104, a concentration of the P-Nu within the aqueous solution 104, a degree of modification of the P-VS, a degree of modification of the P-Nu or a different combination of polymers. For example, the gelation time can be modified so that the gelation time is long enough for a physician to manipulate the aqueous solution 102 at the site/space 104, but short enough to form the hydrogel 106 soon after injection to maintain its shape. The surface properties of the hydrogel can be similarly modified. Each property can be adjusted independently.

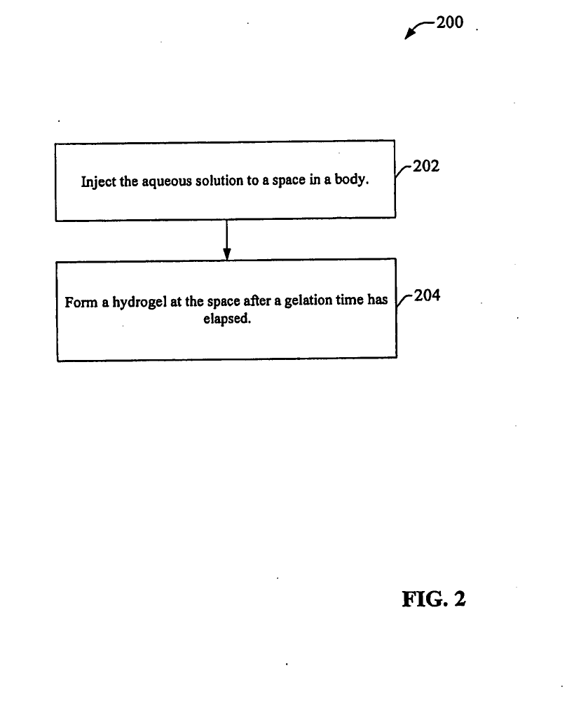

Referring now to FIG. 2, illustrated is an example non-limiting process flow diagram of a method 200 for facilitating the formation of a hydrogel at a site in the body, according to an embodiment. The method 200 facilitates the formation of an “injectable hydrogel,” a hydrogel that can be assembled in situ upon delivery to a site in the body at physiological conditions.

In an embodiment, the physiological conditions include:

| | |
|---|---|
| Temperature | about 10 degrees Celsius to about 60 degrees Celsius; |
| pH | about 5 to about 9; and/or |
| Water Content | at least about 50 percent. |

In another embodiment, the physiological conditions include:

| | |
|---|---|
| Temperature | about 30 degrees Celsius to about 40 degrees Celsius; |
| pH | about 6.5 to about 8; |
| Water Content | at least about 80 percent. |

In a further embodiment, the physiological conditions include:

| | |
|---|---|
| Temperature | about 35 degrees Celsius to about 39 degrees Celsius; |
| pH | about 7.0 to about 8.0; |
| Water Content | at least about 90 percent. |

At element 202, an aqueous solution containing hydrogel precursors is injected to a spot/site in the body. The aqueous solution is a viscous solution before injection to facilitate the injection. The precursors can include polymers that are biocompatible and non-immunogenic (e.g., do not react with biomolecules, cells, tissues, or the like, related to the injection). Examples of biocompatible polymers whose derivatives can be utilized as precursor molecules include hyaluronic acid (HA), polyethylene glycol (PEG), dextran, carboxymethyl cellulose (CMC), polyvinyl alcohol (PVA), alginate, cyclodextran and the like. In an embodiment, the precursor molecules can be P-VS and P-Nu, as described above with respect to FIG. 1. The aqueous solution can also include a biocompatible buffer solution (e.g., water, PBS, etc.), a salt, an aqueous solvent, any other molecule that regulates the pH of the solution, any other molecule that facilitates modification of the precursor polymer, and therapeutic molecule, or any other molecule that minimizes the potential hazard when the precursors or the hydrogel are used in a biological system.

Properties of the hydrogel, such as mechanical properties, degree of swelling, mesh size, gelation time, degradation time, and the like, can be tailored or engineered independently from each other for different purposes. For example, the properties are controllable based on the pH of the aqueous solution, a temperature of the aqueous solution, a concentration of the P-VS within the aqueous solution, a concentration of the P-Nu containing molecule within the aqueous solution, a degree of modification of the P-VS, a degree of modification of the P-Nu or a different combination of polymers. The surface properties of the hydrogel can be similarly modified.

Upon injection into the site, the aqueous solution can undergo gelation to form a hydrogel. Accordingly, the hydrogel is an in situ chemical crosslinkable formulation. The term in situ chemical crosslinkable formulation generally refers to a hydrogel that can be crosslinked at a condition close to physiological conditions. Accordingly, at element 204, a hydrogel is formed at the space/site after a gelation time has elapsed. For example, the gelation time can be long enough for a physician to manipulate the aqueous solution into a shape at the site/space, but short enough to form the hydrogel soon after injection to maintain its shape.

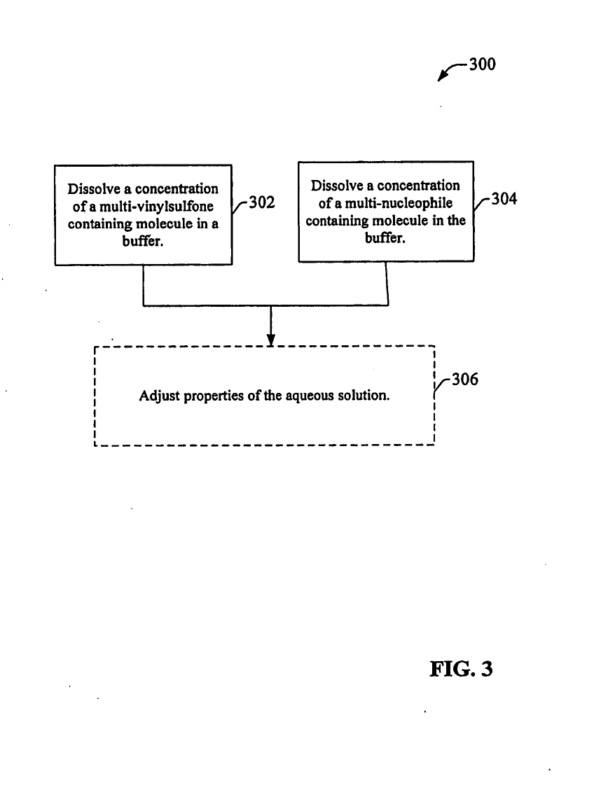

Referring now to FIG. 3, illustrated is an example non-limiting process flow diagram of a method 300 for forming the aqueous solution that can facilitate the formation of a hydrogel at a site/space in the body, according to an embodiment.

At element 302, a concentration of P-VS is dissolved in a biocompatible buffer (e.g., water, PBS, or the like). The polymer that is modified with vinylsulfone can be biocompatible, non-immunogenic, and soluble in the buffer.

At element 304, a concentration of P-Nu is dissolved in the biocompatible buffer. The polymer that is modified with the nucleophile can be biocompatible, non-immunogenic, and soluble in the buffer. In an embodiment, the polymer that is modified with the nucleophile can be of the same polymer backbone as the polymer that is modified with the vinylsulfone.

Optionally, at element 306, properties of the aqueous solution can be adjusted to facilitate adjustment of properties and/or characteristics of the hydrogel that will be formed upon injection to a site/space in the body. For example, the pH of the aqueous solution, a temperature of the aqueous solution, a concentration of the P-VS within the aqueous solution, a concentration of the P-Nu containing molecule within the aqueous solution, a degree of modification of the P-VS, a degree of modification of the P-Nu or a different combination of polymers can be adjusted to facilitate modification and adjustment of the properties and/or characteristics of the hydrogel. Properties and/or characteristics of the hydrogel include: surface properties, mechanical properties, degree of swelling, mesh size, gelation time, degradation time, and the like.

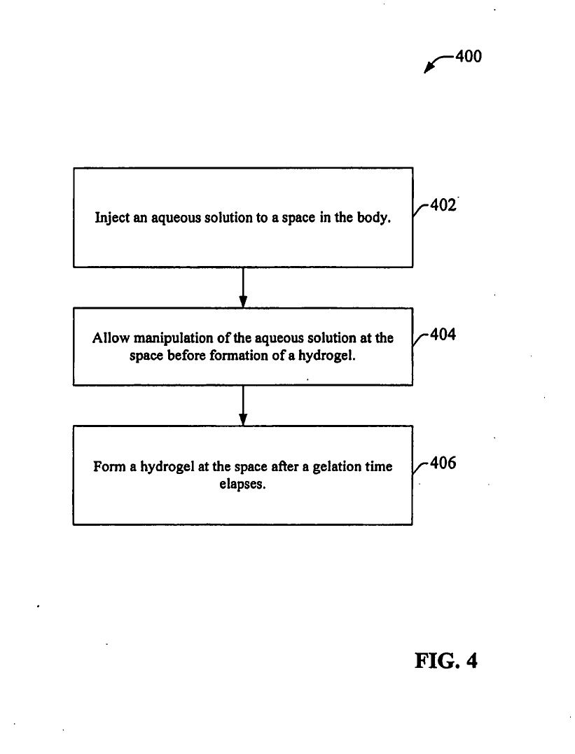

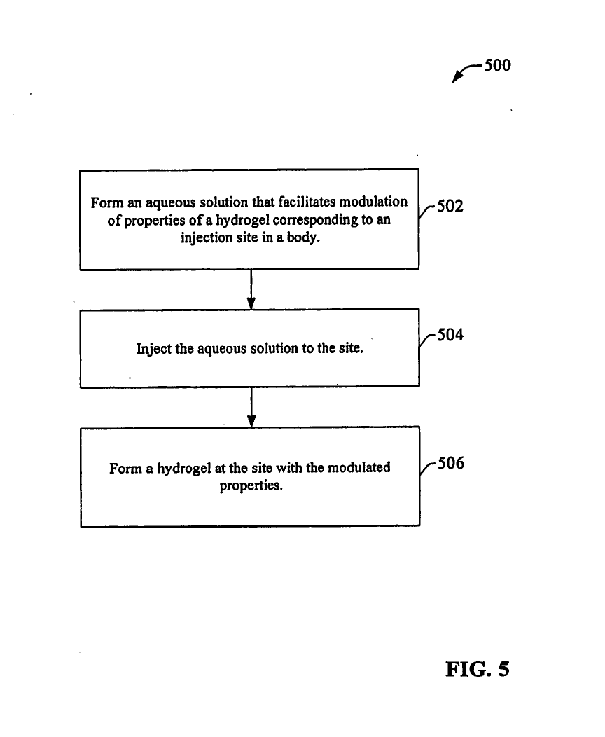

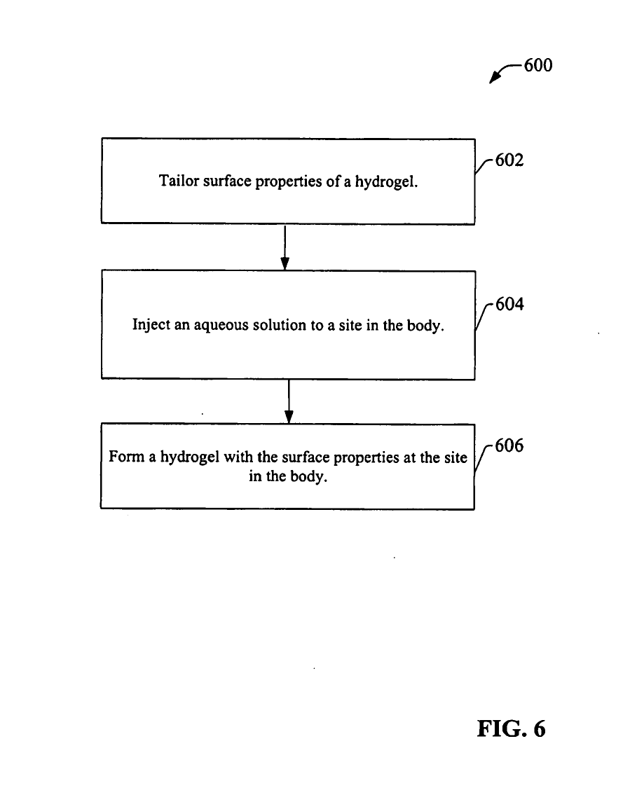

The injectable hydrogel, as described, for example in FIGS. 1-3, that can form in situ with controllable properties can be used in many biomedical applications. The injectable hydrogel can be injected to the site/space with a small-gauge needle (about 30 gauge or smaller sized) and has properties and/or characteristics that can be chemically controlled to facilitate in situ formation of the hydrogel. Examples of biomedical applications include, but are not limited to, a volume/filler expander (FIG. 4), a storage and delivery mechanism for biologics (FIG. 5), and as a tissue interacting surface (FIG. 6).

Referring now to FIG. 4, illustrated is an example non-limiting process flow diagram of method 400 for utilizing an injectable hydrogel as a volume filler/expander, according to an embodiment. A volume filler/expander can find use in cosmetic, reconstructive and plastic surgery, for example, as an implant material for soft tissue augmentation (such as anti-wrinkle treatment, breast augmentation, and the like), body volume restoration, and the like. For these applications, the implant material should be biocompatible and non-immunogenic, should provide a long-lasting effect, and should be able to be administered to the site/space by a minimally invasive method without migration of the material. In certain circumstances, the material should also be removable.

A potential candidate for such material is a HA-based soft tissue augmentation material. Although unmodified HA can be degraded quickly in a human, when HA is chemically crosslinked to form a hydrogel, the degradation can be sharply reduced. For this reason, crosslinked HA has been widely used as a soft tissue augmentation device or as a dermal filler.

Conventionally, the crosslinking reactions are conducted in vitro. Commercial products (e.g., RESTYLANE® and JUVEDERM®) utilize a preformed hydrogel. The preformed hydrogel often includes ground HA hydrogel particles embedded in a high molecular weight HA viscous solution (called HA “slurry”).

There are several disadvantages associated with the HA slurry approach. First, the injected material may dislocate after injection. Because in the slurry the crosslinked HA particles are only mixed with a non-crosslinked high molecular weight HA solution, when the HA solution flows in response to stress, the particle may be carried to an undesirable location. Secondly, because the HA solution will be absorbed by the body shortly after injection, to restore the lost volume from uncrosslinked HA, the particles need to be able to swell to take up the space. A high degree swelling requires low degree of crosslinking; however, low degree of crosslinking increase the rate of degradation. Thirdly, because the upper dermis composes of densely packed collagen fiber, smaller gauge needle (e.g., 30 gauge or larger in other words, a size smaller than or equal to 30 gauge) has to be used to generate enough pressure to push the slurry through the epidermis, but the smaller needle only allows smaller particles to go through; however, smaller particle size leads to faster degradation.

Method 400, employs an entirely different approach to facilitate volume filling and expansion. At element 402, an aqueous solution is injected to a space in the body. The space can be the area in need of volume filling or expansion. The aqueous solution can include an aqueous buffer and gel precursors dissolved in the aqueous solution (e.g., P-VS and P-Nu as described with respect to FIG. 1). Also dissolved in the aqueous solution can be additives that can facilitate control of parameters and/or characteristics of the hydrogel. The aqueous solution is a viscous solution so that it can be easily injected.

The hydrogel can be formed inside the body upon injection at a biological pH. At element 404, the aqueous solution can be manipulated at the space before the gel is formed (before gelation). The gelation time, for example, can be long enough for a physician to manipulate the aqueous solution into a shape, but short enough to facilitate formation of a gel soon after injection so that the gel can maintain its shape. At element 406, after the gelation time has elapsed, the hydrogel can be formed at the space according to the manipulation. After gelation, the hydrogel is a bulk hydrogel instead of particles, so that the total surface area is smaller and the degradation rate is reduced. The degree of crosslinking, which is related to the degree of modification and the concentration, can be relatively high so that the degradation rate can be further reduced.

Similarly, method 400 can also be utilized to fill an aneurism bulge. An aneurism is a cardiovascular disease that affects the peripheral and/or cerebral blood vessels where the blood vessel, usually an artery, develops a localized blood-filled balloon-like bulge, which has a fragile blood vessel wall. Patients developing an aneurism carry the risk of rupture of the bulge and subsequent hemorrhage, which may lead to severe complications, including death. One way to treat an aneurism is to isolate the bulge space from the blood circulation by filling the bulge, so that the risk of rupture will, thus, be reduced. However, the material previously available was limited and often associated with unsatisfactory clinical outcome.

The material must be able to pass through a long and thin microcatheter so that the liquid should not be too viscous and the gelation cannot be too rapid. After the material arrives at the bulge, it should stay in the bulge. The material should have some anti-fouling properties, such that the proteins in the blood will not accumulate on the surface and block the blood vessel. Moreover, the material should be long-lasting.

The injectable hydrogel described herein has characteristics suitable as a material for treating aneurisms. The viscosity can be controlled to be low enough to go through a thin microcatheter. The gelation time can be controlled so that the polymer solution can travel through the microcatheter and form a gel after arriving at the bulge. The swelling of the gel can be controlled so that it can better stay in the bulge by physical expansion. The surface of the gel can be engineered to be anti-fouling. Moreover, the degradation time of the gel can be controlled.

Referring now to FIG. 5, illustrated is an example non-limiting process flow diagram of a method 500 for utilizing an injectable hydrogel to facilitate biologic storage and/or delivery, according to an embodiment. With the advance of biomedical engineering, biologic therapeutic agents, including peptides, protein, nucleic acid and even cells, has been developed and proved to be effective in treating many diseases. There are two major categories of applications that utilize cell as therapeutic agent. One is in tissue engineering, in which the cells are used to grow new tissues which will be implanted into the patient. To increase the potency, cells are usually dispersed and cultured in a scaffold, and placed at a site of the body where the new tissue is needed. Another approach is to utilize cells' protein-producing machinery, in which cells are engineered to produce certain protein and grow in a scaffold that can be put at a desirable site.

Method 500 illustrates the use of the injectable hydrogel as a cell delivery device. At 502, an aqueous solution is formed that facilitates modulation of properties of a hydrogel corresponding to an injection site in a body. The mechanical properties or the physical microenvironment can be easily modulated to fit different cells and different applications. For example, the polymer can be easily functionalized to contain different biologically active moieties so that the chemical microenvironment can be modulated to fit different cells and different applications.

At 504, the aqueous solution can be injected to the site. The surface of the hydrogel can be easily modified so that it can adhere to the injected site to prevent the loss of cells. At 506, a hydrogel can be formed at the site with the modulated properties. For example, the degradability of the hydrogel can be easily modulated for different applications.

Referring now to FIG. 6, illustrated is an example non-limiting process flow diagram of a method 600 for utilizing an injectable hydrogel as a tissue interacting surface, according to an embodiment. Tissue adhesion after surgery is a common and severe complication in surgery, which may lead to severe pain, inflammation or infertility. The current clinical practice for preventing the adhesion includes using polymer solution, a pre-formed hydrogel, a swellable hydrogel film, a solid sheet or an in situ hydrogel to stop the adhesion.

The polymer solution has the disadvantage that it easily flows away from the injection site so it is not ideal for anti adhesion of a specific operation site. For the pre-formed gel, gel film and solid sheet, complete coverage of the operation site is difficult. The in situ hydrogel is an ideal anti adhesion material because it can cover the whole operated site easily.

According to method 600, the injectable hydrogel can also be used for this purpose. At 602, surface properties of a hydrogel are tailored. Non-surface properties can also be tailored for a specific application. The degradation time of the hydrogel can be tailored so that the hydrogel will stay long enough to prevent adhesion, and eventually gone after the wound is healed. In addition to anti adhesion, the gelation time can be controlled to be very fast (within seconds) such that the gel may be used to stop bleeding.

At 604, an aqueous solution including at least a buffer and the hydrogel precursors can be injected to a site in the body. At 606, a hydrogel is formed with controllable surface properties. The surface of the hydrogel can be engineered such that it will be both compatible with the tissue and preventing tissue from growing on or into the hydrogel.

### EXPERIMENTAL

The following examples are exemplary or illustrative of the application of the principles described above. It will be noted that experimental data provided does not limit the scope of the embodiments. Rather, such data merely illustrate examples of how to adjust the hydrogel properties for different applications. The choices of polymer materials, cell lines, model animals, as well as the detailed experimental procedures, such as pH, osmolality and the like, are merely illustrations.

### Biocompatibility of the Hydrogel

Cell Compatibility

A hydrogel composed of 108 kDa vinylsulfonated hyaluronic acid (HA-VS) and thiolated hyaluronic acid (HA-SH) is used as an example to show the biocompatibility of the hydrogel with regard to cells.

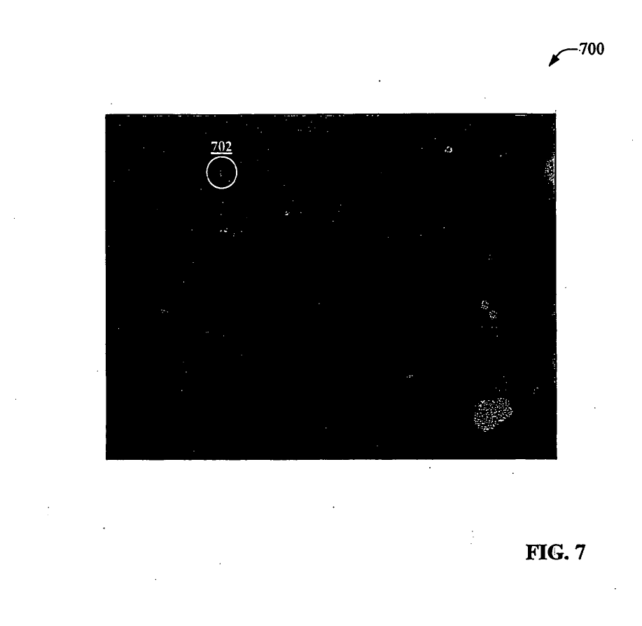

HA-VS and HA-SH of 18% degree of modification (DM) were dissolved in a cell culture medium at 2% w/v. Gels were formed by mixing the two polymers. The gels were placed on top of NIH 3T3 cells, cultured on a 96-well plate, and incubated for 1 day. The cells were stained with Live/Dead assay to examine their viability, as shown in FIG. 7.

Referring now to FIG. 7, illustrated is an exemplary non-limiting representative Live/Dead assay image 700 of NIH 3T3 cells after incubating with hydrogel, according to an embodiment. From the image, it can be determined that the cells are mostly viable. The region labeled 702 includes the only appreciable area of dead cells. Therefore, the hydrogel is generally biocompatible, causing virtually no adverse reactions in the cells.

Cell Encapsulation Compatibility

An experiment was performed to show that cells remain viable after encapsulation within the hydrogel. This property is essential for cell delivery/tissue engineering applications.

A hydrogel composed of 108 kDa HA-VS and HA-SH of 18% DM was used as an example.

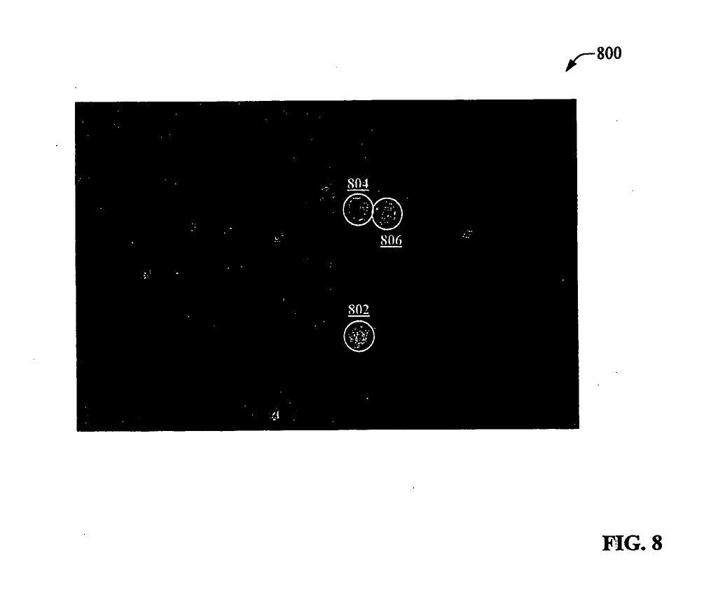

HA-SH was dissolved in a cell culture medium at 2% w/v. HA-SH was first dissolved in the cell culture medium at a higher concentration at a higher concentration and mixed with about 10⁶ cells so that the final concentration also reached 2%. Afterwards, the HA-SH polymer solution and the HA-VS/cell solution was mixed and seeded on a 96-well plate. The cells were cultured in the gel in the presence of cell culture medium for 2 days and were subsequently subjected to a Live/Dead assay as shown in FIG. 8.

Referring now to FIG. 8, illustrated is an exemplary non-limiting representative live/dead assay image 800 of cells cultured within a hydrogel, according to an embodiment. As shown in regions 802, 804 and 806, the cells remain viable when encapsulated in the hydrogel.

In Vivo Compatibility

This example illustrates that the procedure of forming the in situ gel, as well as having prolonged contact with the hydrogel, is compatible with the skin of an animal.

A hydrogel composed of 108 kDa HA-VS and HA-SH of 39% DM was used as an example.

HA-VS and HA-SH were dissolved in PBS at 4% w/v. After mixing, the mixture was loaded on a syringe and injected to SD mice subcutaneously with a 30 gauge needle.

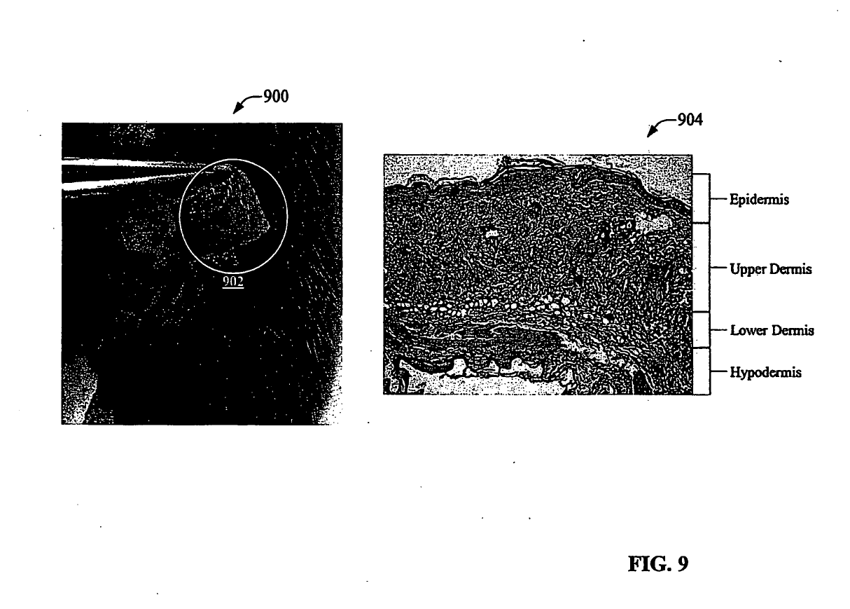

FIG. 9 is an exemplary non-limiting illustration showing that mouse skin cells tolerate the hydrogel, according to an embodiment. The mice were sacrificed after 3 weeks (image 900). The skin tissue adjacent to the gel 902 was fixed and stained with H & E stain (image 904). As shown in image 904, the mouse skin cells are tolerant to the gel.

### Controlling the Gelation Time of the Hydrogel

The gelation time of the hydrogel can be controlled by many factors. Examples of these factors include: reaction pH, temperature, choice of polymer, polymer concentration, degree of modification, and the like.

HA-VS and HA-SH, vinylsulfonated dextran (dex-VS), thiolated dextran (dex-SH) and vinylsulfonated polyvinyl alcohol (PVA-VS) were used as examples. The polymers were dissolved in a buffer solution and were mixed with a counterpart to from a hydrogel. The gelation time is defined by a tube inversion test. If not specified, the pH of the polymer solution is kept at 7.4.

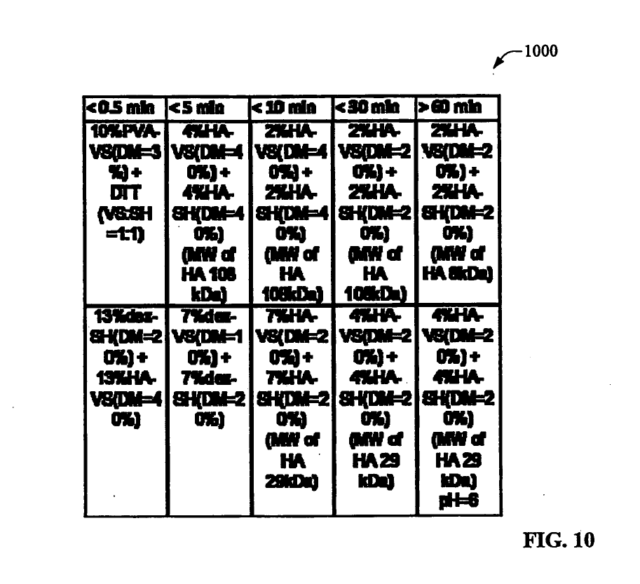

Referring now to FIG. 10, illustrated is an exemplary non-limiting table 1000 illustrating the possibility of achieving different gelation times using different combinations of parameters, according to an embodiment. It should be noted, however, that the formulations under each gelation time category is served only as an example. Many additional possibilities exist. The choice of appropriate conditions depends on the specific application.

### Controlling Swelling

In Vitro Swelling Tests

The swelling ratio is defined as the volume of gel after swelling over the volume of gel before swelling. By controlling the degree of modification and the concentration of polymer, the swelling of the hydrogel can be controlled.

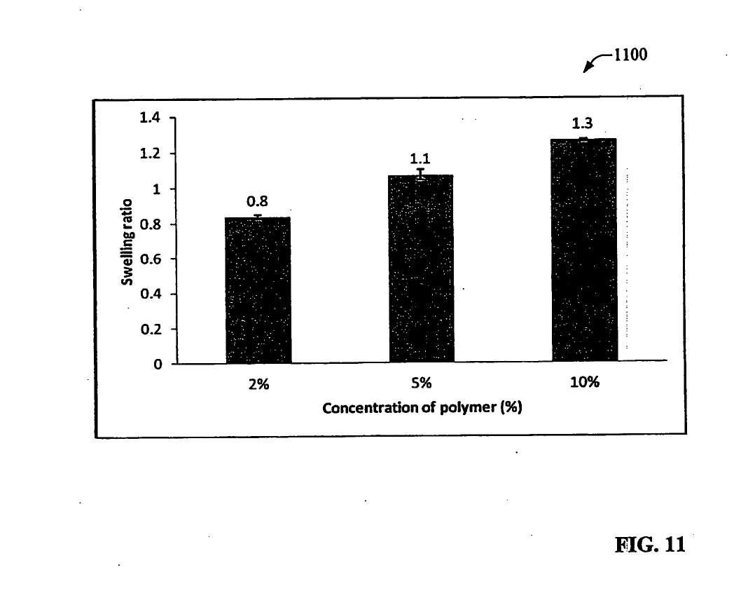

HA-VS and HA-SH of DM 20% is used as an example to show that the swelling ratio can be controlled to be around 1 (as shown in FIG. 11).

HA-VS and HA-SH were dissolved in PBS. Polymer counterparts of the same concentration were mixed and form the hydrogel. The hydrogel was then swelled in PBS to reach equilibrium swelling. The weight of the hydrogel before and after the swelling were measured and used to calculate the volume change, assuming the volume of the hydrogel is the sum of the volume of the polymer and the volume of the PBS.

Referring now to FIG. 11, illustrated is an exemplary non-limiting illustration 1100 of the swelling of a hydrogel made from mixing HA-VS and HA-SH of DM 20%, according to an embodiment.

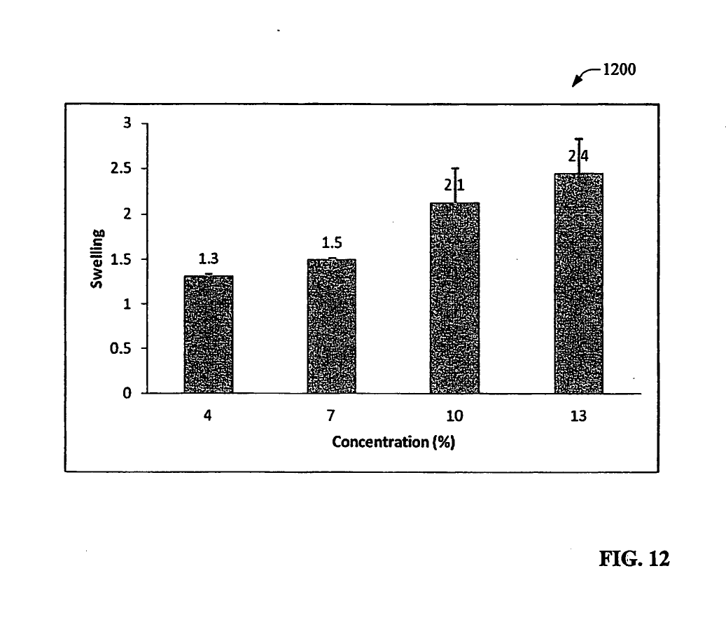

In another example, HA-VS of DM 8% and dex-SH of DM 4% are used to show that the swelling ratio can be controlled to be around 2 (as shown in FIG. 12).

Polymer counterparts of the same concentration were mixed to form the hydrogel. The hydrogel was then swelled in PBS to reach equilibrium swelling. The weight of the hydrogel before and after swelling were measured and used to calculate the volume change, assuming the volume of the hydrogel is the sum of the volume of the polymer and the volume of PBS.

Referring now to FIG. 12 is an exemplary non-limiting illustration 1200 of the swelling of a hydrogel made from mixing HA-VS of DM 8% and dex-SH of DM 4%, according to an embodiment.

In Vivo Expansion

This example intends to show that the in situ hydrogel can create a new volume (as opposed to diffusing away from the injection site) after injection to the body. The new formulations also have better control of swelling and longer augmentation effect than existing dermal filters (e.g., JUVERDERM® and RESTYLANE®).

To evaluate the in vivo augmentation effect of the hydrogel, two formulations with different mechanical properties (˜1000 Pa and ˜6000 Pa storage modulus) were injected to mice subcutaneously. The sizes of the hydrogels were measured using a caliper.

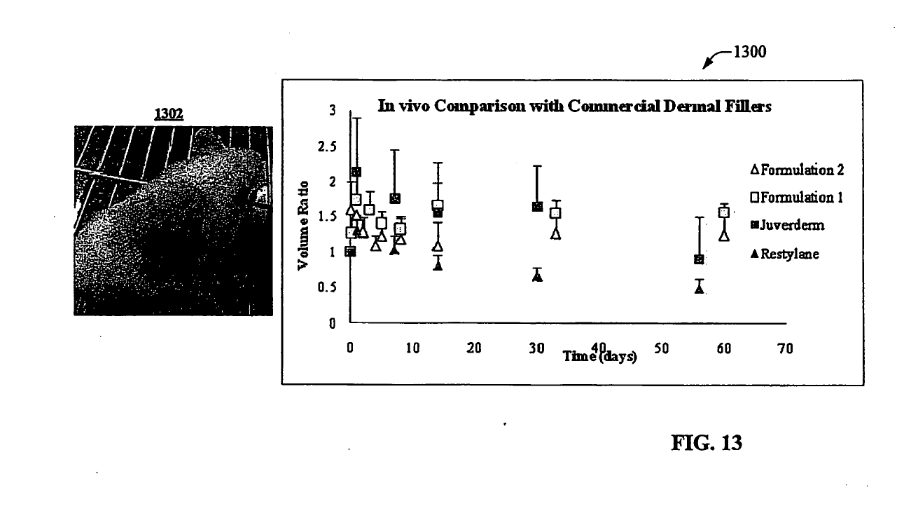

It was shown that the gel can indeed expand the volume after injection. An image illustrating the swelling is shown at element 1302 of FIG. 13.

FIG. 13 is an exemplary non-limiting graph 1300 illustrating in vivo swelling and longevity of two formulations compared with commercially available fillers, according to an embodiment. Compared with existing in vivo augmentation effect data on JUVERDERM® and RESTYLANE® in rodent (as shown by Hillel, et al. Dermatological Surgery 38, 471-478, 2012), both of the hydrogel formulations have better control of swelling (smaller error bar) and slower degradation.

### Controlling Mesh Size

In Vitro Characterization of Mesh Size

This example illustrates that the mesh size of the hydrogel network can be controlled by varying the degree of modification and the polymer concentration.

The mesh size can be calculated according to Peppas' iconic polymer swelling model:

$$\frac{{V}_{1}}{4 l} {( \frac{{υ}_{2 , s}}{\overline{υ} {M}_{0}} )}^{2} {( \frac{{10}^{- {pK}_{a}}}{{10}^{- p H} + {10}^{{pK}_{a}}} )}^{2} = ln ( 1 - {υ}_{2 , s} ) + {υ}_{2 , s} + {χ}_{1} {υ}_{2 , s}^{2} + ( \frac{{V}_{1}}{\overline{υ} {M}_{c}} ) ( 1 - \frac{2 {M}_{c}}{{M}_{n}} ) {υ}_{2 , r} [ {( \frac{{υ}_{2 , s}}{{υ}_{2 , r}} )}^{1 / 3} - \frac{1}{2} ( \frac{{υ}_{2 , s}}{{υ}_{2 , r}} ) ]$$

The first example utilizes HA-VS of DM 8% and dex-SH of DM 4% as precursor polymers. The polymers were dissolved in PBS, and polymer counterparts of the same concentration were mixed to form a hydrogel. The hydrogels were swollen in PBS and the weight of pre-swollen hydrogels, swelled hydrogels, and dried polymer after swelling were measured and used to calculate the mesh size.

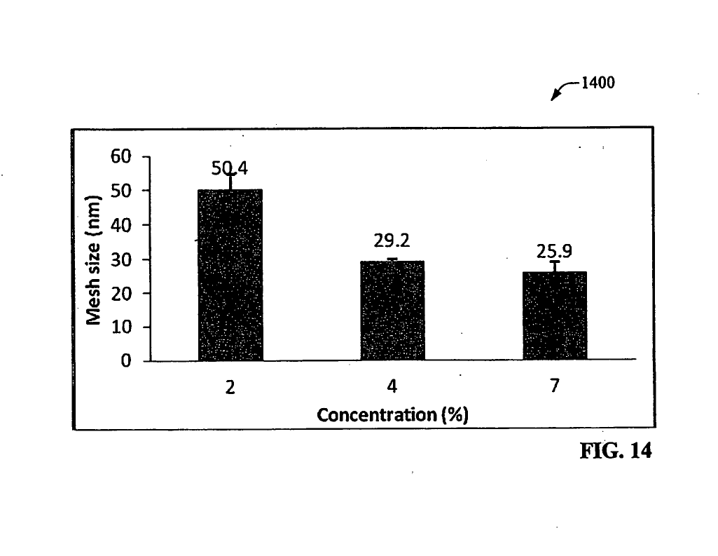

FIG. 14 shows an exemplary non-limiting illustration 1400 of the control of a mesh size of a hydrogel made from mixing HA-VS of DM 8% and dex-SH of DM 4%, according to an embodiment. As shown in FIG. 14, the mesh size can e controlled from 50 nm to 25 nm.

The second example uses HA-VS and HA-SH of DM 20% as precursor polymers. The polymers were dissolved in PBS and polymer counterparts of the same concentration were mixed to form the hydrogel. The hydrogel was swollen in PBS and the weight of the pre-swollen hydrogel, swollen hydrogel, and dried polymer were measured and used to calculate the mesh size.

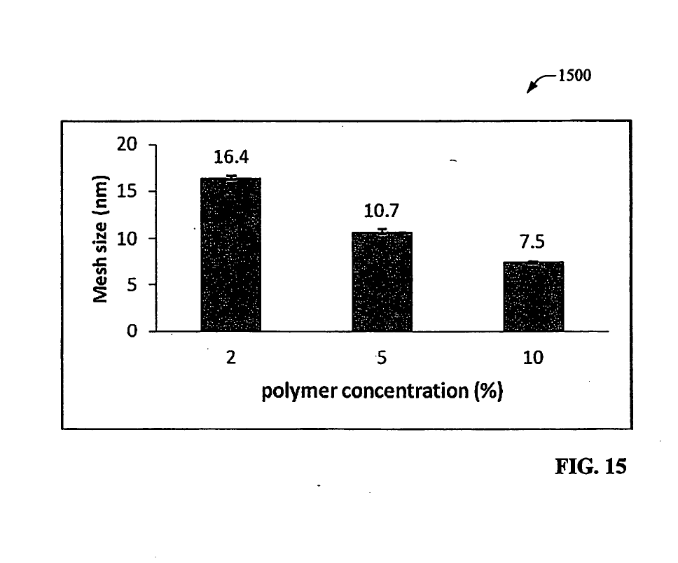

FIG. 15 shows exemplary non-limiting illustration 1500 of the control of a mesh size of a hydrogel made from mixing HA-VS and HA-SH of DM 20%, according to an embodiment. As shown in FIG. 15, the mesh size could be controlled from 16 nm to 7.5 nm.

In Vivo Protein Controlled Release from Hydrogel

The ability to control the mesh size enables better control so that the hydrogel can be utilized as a biologics storage and delivery device. The following example shows that a protein can be encapsulated within the hydrogel and controlled release of protein can be achieved for a prolong period of time.

A Hydrogel composed of 29 kDa HA-VS and HA-SH of 20% DM was used as an example. Near infrared labeled IgG (NIR-IgG) was used as a model protein.

HA-VS and HA-SH were dissolved in PBS at 10% w/v. The two polymers were mixed together with NMR-IgG, and injected subcutaneously to the mice.

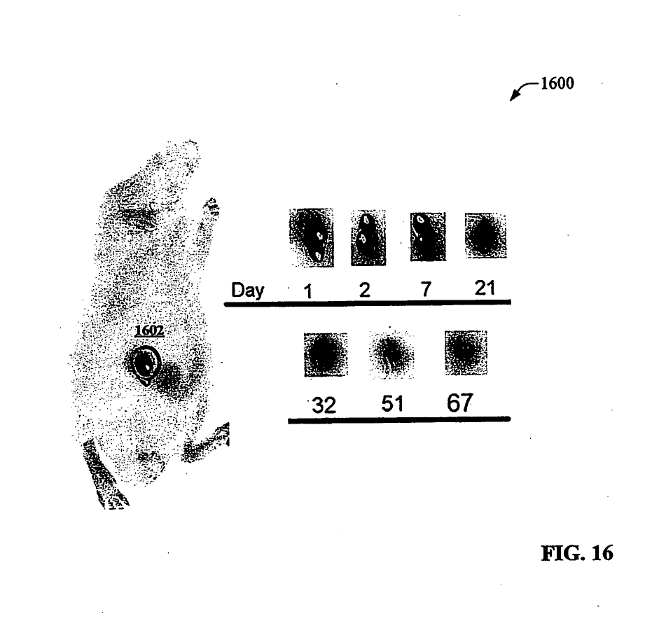

The presence of NIR-IgG in the hydrogel was visualized by NIR scanner. FIG. 16 is an exemplary non-limiting NIR image 1600 of NIR-IgG encapsulated hydrogel injected subcutaneously 1602 into a mouse, according to an embodiment. The result shows that the protein can be encapsulated and the release can be delayed to more than 2 months

### Controlling the Degradation of the Hydrogel

The following examples illustrate the control the degradation of the hydrogel.

The hydrogel in the body may be degraded by hydrolysis or enzyme. HA is used as an example polymer for degradation control because of the ubiquitous presence of hyaluronidase in the body.

A hydrogel composed of 108 kDa HA-VS and HA-SH of 18% DM was used as an example. The polymers were dissolved in PBS and mixed to form a hydrogel. The gels were then incubated in PBS containing 50 U/ml hyaluronidase.

After one day, the gel was removed and dried, and the dry weight of polymer was counted as the “insoluble fraction” and compared with published data of a dermal filler hydrogel

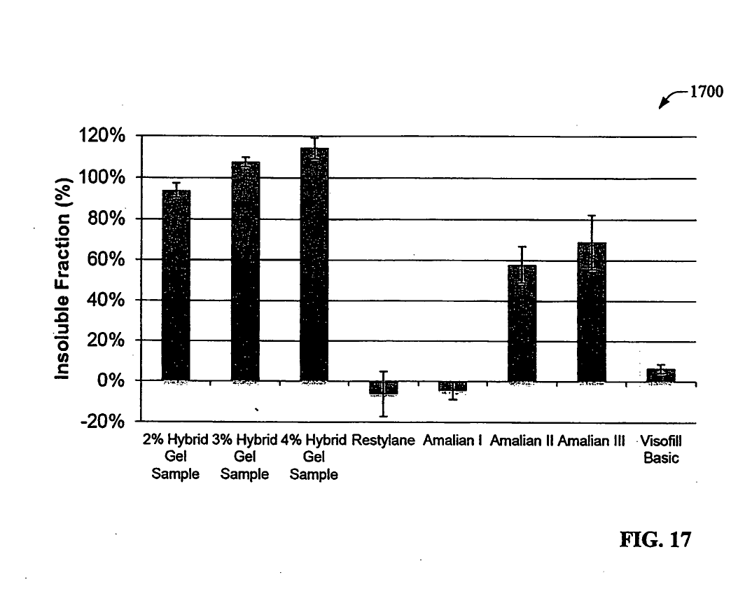

FIG. 17 is an exemplary non-limiting illustration 1700 comparing the degradation of HA based in situ hydrogels compared to commercially available dermal filter hydrogels, according to an embodiment. The degradation of HA based in situ hydrogel is compared to commercially available dermal filler hydrogels (Restylane, Amalian, and Visofill Basic). The result shows that the degradation of the hydrogel can be controlled by varying the HA concentration. The degradation rate can be far slower than commercially available dermal filler hydrogels.

### Controlling the Mechanical Properties of the Hydrogel

A hydrogel composed of 29 kDa HA-VS and HA-SH of 20% DM was used as an example to show the control of mechanical properties.

A dynamic mechanical analyzer (DMA) was used to measure the storage modulus (G′) of hydrogel of different polymer concentrations.

PBS was used as solvent for all polymers. The polymers were mixed with their counterparts of the same concentration, and immediately loaded on the DMA machine and the measurement started.

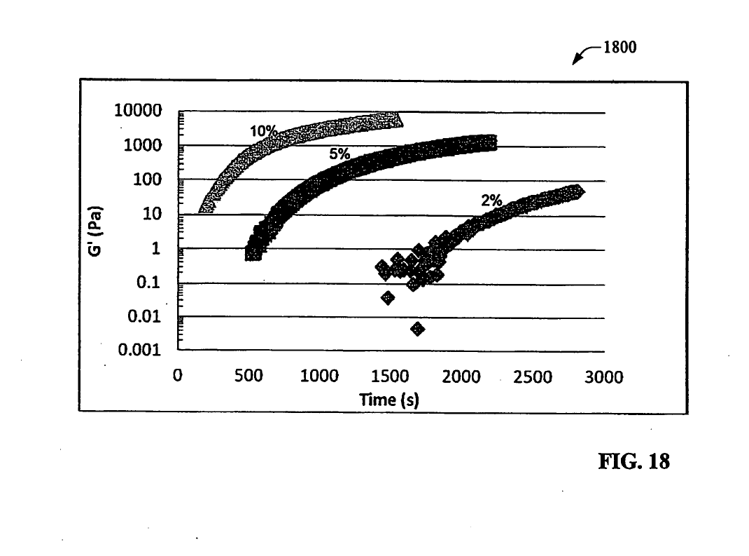

FIG. 18 is an exemplary non-limiting graph 1800 showing the storage modulus of hydrogels of different compositions (2%, 5%, 10%), according to an embodiment. The results shown in FIG. 18 confirm that the mechanical properties of the hydrogel can be controlled from about 100 Pa to at least 10 kPa.

### Modifying the Surface Properties of the Hydrogel

The following examples show that the surface properties of a hydrogel can be modified by selecting a specific polymer and chemically functionalizing the polymer.

Making an Anti-Adhesion Hydrogel

An HA based hydrogel, because of its anti-adhesion properties, is used as an example.

108 kDa HA-VS and HA-SH of 18% DM were dissolved in tissue culture medium at 2% w/v and mixed to form a hydrogel. About 10⁴ cells were seeded on top of the hydrogel.

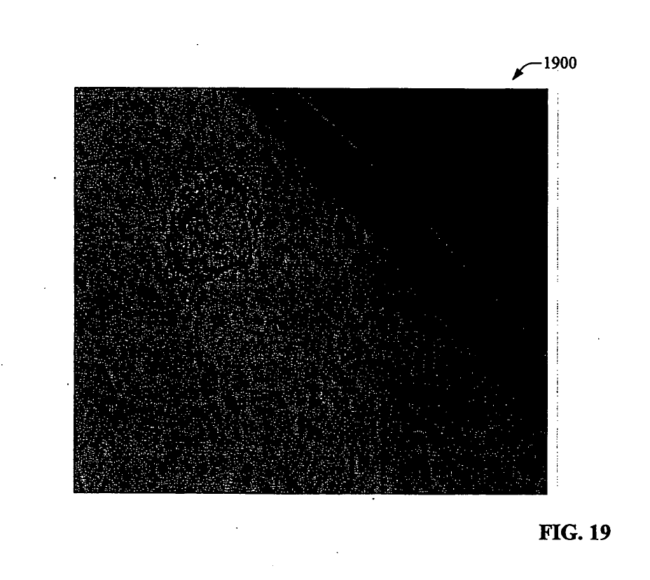

A light microscope was used to examine the morphology of cells and a live/dead assay was used to examine viability of cells after one day. FIG. 19 is an exemplary non-limiting representative live/dead assay image 1900 of a cell cluster on a HA based hydrogel after one day, according to an embodiment. The cells remain viable, but do not adhere to the hydrogel and form a cluster.

### Biological Signal Functionalization

Peptides and proteins are the basis of biological signals. The vinylsulfonated polymer can be functionalized with cysteine containing protein or peptides.

The following example shows how to make in situ protein conjugated hydrogel. HA-based hydrogel and cysteine containing protein bocine serum albumin was used as a model protein. 29 kDa HA-VS and HA-SH of DM 20% were dissolved in PBS and mixed together with BSA to form a hydrogel. The BSA was found to be conjugated on the hydrogel.

What has been described above includes examples of the embodiments of the subject disclosure. It is, of course, not possible to describe every conceivable combination of components or methods for purposes of describing the claimed subject matter, but it is to be appreciated that many further combinations and permutations of the various embodiments are possible. Accordingly, the claimed subject matter is intended to embrace all such alterations, modifications, and variations that fall within the spirit and scope of the appended claims. While specific embodiments and examples are described in this disclosure for illustrative purposes, various modifications are possible that are considered within the scope of such embodiments and examples, as those skilled in the relevant art can recognize.

In addition, the words “example” or “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

In addition, while an aspect may have been disclosed with respect to only one of several embodiments, such feature may be combined with one or more other features of the other embodiments as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

Numerical data, such as temperatures, concentrations, times, ratios, and the like, are presented herein in a range format. The range format is used merely for convenience and brevity. The range format is meant to be interpreted flexibly to include not only the numerical values explicitly recited as the limits of the range, but also to include all the individual numerical values or sub-ranges encompassed within the range as if each numerical value and sub-range is explicitly recited. When reported herein, any numerical values are meant to implicitly include the term “about.” Values resulting from experimental error that can occur when taking measurements are meant to be included in the numerical values.

What is claimed is:

1. A material, comprising:
a multi-vinylsulfone containing molecule; and
a multi-nucleophile containing molecule,
wherein the material dissolves in a buffer solution to form an aqueous solution, and
wherein the aqueous solution undergoes gelation upon administration to a space in a body with at least one of a controllable gelation time, a controllable swelling ratio, a controllable degradation time and a controllable mechanical property to facilitate filling the space.

2. The material of claim 1, wherein the gelation time is controlled based on a pH of the aqueous solution, a temperature of the aqueous solution, a concentration of the multi-vinylsulfone containing molecule within the aqueous solution, a concentration of the multi-nucleophile containing molecule within the aqueous solution, a degree of modification of the multi-vinylsulfone containing molecule, a degree of modification of the multi-nucleophile containing molecule, or a combination of different molecules.

3. The material of claim 1, wherein the swelling ratio is controlled based on a pH of the aqueous solution, a temperature of the aqueous solution, a concentration of the multi-vinylsulfone containing molecule within the aqueous solution, a concentration of the multi-nucleophile containing molecule within the aqueous solution, a degree of modification of the multi-vinylsulfone containing molecule, a degree of modification of the multi-nucleophile containing molecule, or a combination of different molecules.

4. The material of claim 1, wherein the degradation time is controlled based on a pH of the aqueous solution, a temperature of the aqueous solution, a concentration of the multi-vinylsulfone containing molecule within the aqueous solution, a concentration of the multi-nucleophile containing molecule within the aqueous solution, a degree of modification of the multi-vinylsulfone containing molecule, a degree of modification of the multi-nucleophile containing molecule, or a combination of different molecules.

5. The material of claim 1, wherein the mechanical property is controlled based on a pH of the aqueous solution, a temperature of the aqueous solution, a concentration of the multi-vinylsulfone containing molecule within the aqueous solution, a concentration of the multi-nucleophile containing molecule within the aqueous solution, a degree of modification of the multi-vinylsulfone containing molecule, a degree of modification of the multi-nucleophile containing molecule, or a combination of different molecules.

6. The material of claim 1, wherein the aqueous solution undergoes the gelation when the multi-vinylsulfone containing molecule forms a covalent bond with the multi-nucleophile containing molecule.

7. The material of claim 1, wherein the multi-vinylsulfone containing molecule comprises a polymer comprising at least two vinylsulfone groups.

8. The material of claim 1, wherein the multi-nucleophile containing molecule comprises a polymer comprising at least two nucleophile groups.

9. The material of claim 1, wherein the multi-nucleophile containing molecule comprises a thiol or an amine.

10. The material of claim 1, wherein the aqueous solution further comprises a salt, an organic solvent, or a therapeutic agent.

11. A method, comprising:
dissolving a multi-vinylsulfone containing molecule and a multi-nucleophile containing molecule in water to form an aqueous solution;
injecting the aqueous solution to a space in a body; and
forming a hydrogel at the space after a gelation time elapses to facilitate filling the space.

12. The method of claim 11, further comprising controlling the gelation time of the hydrogel, a swelling ratio of the hydrogel, a degradation time of the hydrogel or a mechanical property of the hydrogel.

13. The method of claim 11, wherein the forming further comprises covalently bonding the multi-vinylsulfone containing molecule to the multi-nucleophile containing molecule.

14. The method of claim 13, wherein the covalently bonding further comprises covalently bonding the multi-vinylsulfone containing molecule to the multi-nucleophile containing molecule with at least one of a pH of about 5 to about 9, a temperature of about 10 degrees Celsius to about 60 degrees Celsius or a water content of at least about 50 percent.

15. The method of claim 11, wherein the injecting further comprises injecting the aqueous solution to the space with a needle size of about 30 gauge or smaller sized.

16. The method of claim 11, further comprising manipulating the aqueous solution at the space before the hydrogel is formed.

17. A method, comprising:
forming an aqueous solution by dissolving a multi-vinylsulfone containing molecule and a multi-nucleophile containing molecule in water;
administering the aqueous solution to a site in a body; and
creating a biocompatible surface that interacts with the body upon administration to the site.

18. The method of claim 17, wherein the biocompatible surface does not support neighboring cells growing in or onto the surface.

19. The method of claim 17, wherein the biocompatible surface attaches adheres to neighboring tissue.

20. The method of claim 17, wherein the biocompatible surface supports healthy growth of neighboring tissue.

21. The method of claim 17, further comprising controlling the gelation time of the hydrogel, a swelling ratio of the hydrogel, a degradation time of the hydrogel or a mechanical property of the hydrogel.
